(19) United States
(12) Patent Application Publication
(10) Pub. No.: US 2012/0009641 A1
(43) Pub. Date: Jan. 12, 2012

(54) METHOD FOR PRODUCING CLARIFIED JUICE, PACKAGIBLE JUICE, ETHANOL AND SUGAR FROM SUGARCANE AND SYSTEM THEREOF

(76) Inventors: Vishnukumar Mahadeo Kulkarni, Maharashtra (IN); Umesh Venkatesh Kulkarni, Maharashtra (IN)

(21) Appl. No.: 13/257,642

(22) PCT Filed: Mar. 25, 2010
(86) PCT No.: PCT/IN10/00186

§ 371 Date: Sep. 20, 2011

(30) Foreign Application Priority Data
Mar. 26, 2009 (IN) 733/MUM/2009

(51) Int. Cl. C12P 7/06; A23L 1/277; A23L 1/00; A23L 2/38; C12M 1/00; C13B 10/14

(52) U.S. Cl. 435/161; 435/289.1; 127/44; 99/510; 426/271; 426/253

(57) ABSTRACT

The present invention provides a method and system for manufacturing clarified juice from sugarcane without using sulfitation process and with less heating. The method comprises step of steps of: washing sugarcane with water comprising biocide before passing through cutter and shredders for removing mud and reducing microbial population; spraying biocide on the cut and prepared sugarcane to reduce the microbial activity; adding biocide while milling of the sugarcane or in juice collectors to have control over the microbial growth; filtering the juice to remove floating/suspended matters; heating the filtered juice at about 35-85 deg C.; mixing one or more coagulating and flocculating agents for coagulation of suspended solids; and separating the coagulated solids to obtain clarified juice. This juice can be further treated to manufacture ethanol, juice for packaging having shelf life of about six months and sugar without using conventional sulfitation treatment.

### FIELD OF THE INVENTION

This invention relates to a method of producing clarified juice from sugarcane, packagible sugarcane juice and fermenting the juice to produce ethanol or sugar and the systems thereof.

### BACKGROUND ART OF THE INVENTION

Sugarcane juice is most nutritive drink that contains lots of minerals essential for life. Sugarcane juice is not only used for drinking purpose but also use for producing ethanol and sugar.

In sugar manufacturing process, sugarcane juice is extracted by milling after cutting and shredding the sugarcane and then mixed juice is formed. This mixed juice contains lots of bagasse/bagacillo (fine particles of bagasse), mud from farm carried along with the sugarcane, and other organic and inorganic colloidal and soluble impurities with sucrose. This mixed juice usually has acidic pH about 5.5 or less. This acidity of the juice depends on cane variety, maturity, freshness and extent microbial infection to the sugarcane from harvesting to milling. This mixed juice after various clarification treatments passed through the series of juice heater to raise the temperature of the juice above 60-70° C. Lime is injected in the heated juice to raise the pH of the juice to 8.5-9.5 and passed through the sulfiter for treating with SO₂. The neutral sulfited juice is then passed through another set of heaters to heat the juice above about 115° C. This heated juice is then sent to the Dorr clarifier where settling aids/flocculating agents may be added to promote juice clarification. The overflow of clarifier called as clarified juice is then evaporated in series of juice evaporators to about 50-60% concentration, called as syrup; which is then processed in A, B, C mesquite (pan) boiling system well known to sugar technologists to produce crystalline sugar. The sugar obtained from A and B mesquite boiling system is forwarded to packing whereas the sugar obtained from C mesquite boiling system is generally re-circulated to A and B mesquite boiling system. This sugar is a double cured sugar and comprises lots of impurities (purity about 94 pol) and has color of about 1000-2000 IU, melting of such sugar increases recirculation of impurities and thus color and quality of sugar is affected. The mud form the bottom of the clarifier is taken to Oliver filter to separate mud as filter cake and filtered juice is returned to the process either in mixed juice or in clear juice or as per demand of the process as decided by chemists. This filtered juice also has lots of impurities and is known as one of the major reasons of impurity rise in the sugar.

The products of sugar processing industries are sugar, and molasses and the other products are mud/filter cake, bagasse. The molasses contains sugars and remaining impurities. This molasses is used to produce alcohol by fermenting the molasses using yeast. However, the molasses quantity is not sufficient to produce required quantity of alcohol and thus there is need to develop viable technology to produce alcohol from other sources like sweet sorghum juice, secondary juice or even sugarcane juice directly or other in-house process raw materials.

The problems in the present sugar manufacturing process are as follows:
Ingression of impurities and micro organisms when the cane is cut for harvesting and transported;
Unhindered microbial growth at every stage of sugar juice processing;
Retention time of 45 to 200 minutes is required in present clarification system depending on the design of the clarifiers (Dorr clarifier.
Clarification process practiced currently is known to increase calcium content of the clarified juice. This would increase scaling problems for distillation column and reduces heat transfer there by increasing cost of distillation. Presence of organic matter worsens the scaling problem and makes cleaning of scales significantly difficult
During clarification, temperature is required to be maintained which consumes energy, especially at Dorr clarifier temperature must be above 105° C.
High temperature destroys reducing sugars that may lead in increase in the color.
High temperature favors inversion of sugar, hence lot of sucrose is converted to reducing sugars, and quantity varies with retention time, pH and temperature of the clarifier.
Due to very high temperature in Dorr compounded with un-reacted lime and SO₂ molecules, extraction of high molecular weight polymers, organic compounds and/or coloring matter is leached out from bagasse present in the juice. This reduces the overall efficiency of clarification process.
Due to liming and sulfitation steps, calcium content of the juice increase which being non sugar leads in increase in molasses formation and scaling problems and thus increases energy requirement, which further increase chemical consumption required for cleaning leading in decrease in plant life and also reduces sugar quantity as well as quality.
Clarification process used in sugar industry employs heating of the juice above 100° C. leading in removal of essential flavoring compounds and also decrease in the shelf life of the sugar.
Oliver filter too is high power consuming and are just used for dewatering the mud that is coming out of Dorr settler.

The sugar loss takes place at various steps in sugar industry during the treatment over the juice. The steps at which sugar loss takes place are as follows:
During milling due to growth of microorganisms;
Due to heating of juice and treatment of lime to raise pH to alkaline range where reducing sugars are converted to organic acids and then neutralizing it with sulfur dioxide gas;
Maintaining juice above 100° C. for about 2 hours at Dorr clarifier, this causes inversion of sucrose to reducing sugars and also conversion of RS to organic acids. Microbes growing at high temperature also convert sugar to organic acids. This acid increases inversion of sucrose;
During concentration of juice, inversion continues and also growth of microbes growing at high temperature.

Further, there are several impurities present in the sugarcane juice that can adversely affect fermentation. The most important amongst them is microorganisms which affects processing of juice and leads fermentation in many ways:
Microbes degrades sugars at rapid rate to form several metabolites till they are eliminated. Thus more time we take to kill them more are the sugar loss.
Heating of the juice for clarification reduces microbial load substantially, however, does not eliminate it totally.
Due to minute size, the surface to volume ratio of bacteria is more and thus bacterial metabolism is faster than that of yeast.
Presence of these bacteria competes competitively with yeast for sugars present in the fermentation broth. Bacterial growth rate (multiplication) is faster than yeast making lesser sugars available to yeast to produce alcohol, thus alcohol yield is affected adversely.
Microbial metabolites, mostly organic acids being volatile can adversely affect alcohol qualitatively.
Bacterial growth can affect yeast coagulation that can hinder in the distillation process.

Though, sugarcane juice is healthy and nutritive drink, many people hesitate to consume sugarcane juice as contaminated sugarcane juice is also responsible for spreading many gastrointestinal and other diseases. The main reason for this is the fact that sugarcane being most nutritive, it protects and supports the growth of microorganisms, many of them are pathogenic. Many attempts have been made for packaging of the juice however failed as sugarcane juice contains plenty microbes which can grow and spoil the juice attempted to pack.

### SUMMARY OF THE INVENTION

It is the object of the present invention to provide a method for producing sugarcane juice for packaging the juice or for producing the sugar or ethanol, which atleast solves the abovementioned problem.

It is the object of the present invention to provide systems for the methods of the present invention.

Accordingly, the present invention in first embodiment provides a method for producing clarified juice from sugarcane comprising steps of: washing sugarcane with water having biocide wherein the biocide is aqueous formulation of Formula I comprising alone or a mixture of compound selected from a group of ammonium compound, gluteraldehyde or chlorine releasing compounds, hydrogen peroxide, and peroxy acetic acid; passing washed sugarcane through cutter and shredders for removing mud and reducing microbial population; spraying biocide on the cut and prepared sugarcane to reduce the microbial activity wherein the biocide is aqueous formulation of Formula II comprising mixture of synergistically acting solutions of sodium and/or potassium salts of methyl, &/or dimethyl, &/or ethyl &/or cyanodiethyl dithiocarbamates about 25-50% w/w preferably 40±0.5% w/w; one or in combination of amine based, polymer based, phosphate based, phosphonate based, organosulfur based, quinine based chelating/sequestrating/penetrating compound about 0.01 to 5% w/w; and 0 to 10 parts of foaming or non-foaming type dispersant/chelating agent formulated to kill about 90% microbes in one minute; cutting and shredding the sugarcane; adding biocide while milling of the sugarcane or in juice collectors to have control over the microbial growth wherein the biocide added during milling or in the juice collector is aqueous formulation of Formula III comprising: a mixture of synergistically acting solutions of sodium and/or potassium salts of methyl, &/or dimethyl, &/or ethyl &/or cyanodiethyl dithiocarbamates about 25-50% w/w preferably 40±0.5% w/w, and 0 to 10 parts of foaming or non-foaming type dispersant/chelating agent formulated to kill about 90% microbes in ten minute; filtering the juice to remove floating/suspended matters; heating the filtered juice at about 35-85 deg C.; mixing one or more coagulating and flocculating agents for coagulation of suspended solids; and separating the coagulated solids to obtain clarified juice having negligible microbes less than 500 cfu per ml.

In second embodiment, the present invention provides a method for producing ethanol comprising steps of fermenting the clarified juice obtained from the method first embodiment of the present invention by adding fermenting agents such as ethanol, yeast e.g. Saccharomyces spp. etc.

In third embodiment, the present invention provides a method for manufacturing juice for packaging comprises steps of: filtering of clarified juice obtained from first embodiment of the present invention; and pasteurizing and packaging the juice wherein the juice has shelf life about six month as the microbial activity is substantially equal to zero.

In forth embodiment, the present invention provides a method for manufacturing sugar from the sugarcane comprises steps of: adding lime in the clarified juice obtained from the first embodiment of the present invention to raise the pH of the juice in the range of 6-9; heating the juice to flocculate proteins; separating the juice from flocculated proteins; concentrating protein-free juice through settler floater to remove suspended and floating matters; and crystallizing the juice to obtain sugar. Alternatively, the juice from the third embodiment can be used.

In fifth embodiment, the present invention also provides a system for producing a clarified juice from sugarcane comprising: a washing means for washing sugarcane with water and biocide for reducing microbial population; a spraying means for spraying biocides solution on prepared cane before entry to cutter and/or shredder for reducing microbial activity; cutter and shredders for cutting and shredding the sugarcane; a series of mills for extraction of juice form sugarcane; a means for adding biocide in the juice during milling or in juice collectors of the mills; a series of mills for extraction of juice form sugarcane; a means for adding biocide in the juice during milling or in juice collectors of the mills; a gravity filter for filtering the juice; a heater for heating the filtered juice at about 35-85 deg C.; a mixer for mixing coagulating agent and flocculating agent in the heated juice; and a separating means for separating coagulated matters to obtain clarified juice wherein said clarified juice negligible microbes less than 500 cfu per ml.

In sixth embodiment the present invention provides a system for producing juice for packaging comprising a system for producing a clarified juice of fifth embodiment; a filter for filtering the clarified juice; and pasteurizing and packaging means for the filtered juice.

In seventh embodiment, the present invention provides a system for producing a clarified juice as claimed in claim 15 or 16; a series of heater for the heating juice at about 80-100 deg C.; a separating means for separating the heated juice from flocculated proteins; a filtering means for filtering the protein free juice; a concentrating means for concentrating the juice; and a crystallizing means for producing sugar. Alternatively, a system for producing juice for packaging can be used instead of system for clarified juice.

It will be understood that, although the terms first, second, third etc., may be used herein above to describe various embodiments of the invention and these embodiments should not be limited by these terms. These terms are only used to distinguish one embodiment from another embodiment/s. Thus, a first discussed below could be termed a second without departing from the teachings of the present invention.

### BRIEF DESCRIPTION OF THE DRAWINGS

So that the manner in which the above recited features of the present invention can be understood in detail, a more particular description of the invention, briefly summarized above, may be had by reference to various embodiments, some of which are illustrated in the appended drawings. It is to be noted, however, that the appended drawings illustrate only typical embodiments of this invention and are therefore not to be considered limiting of its scope, for the invention may admit to other equally effective embodiments:

FIG. 1 shows a system for producing clarified juice from sugarcane according to the present invention;

FIG. 2 shows a system for producing juice for packaging according to the present invention;

FIG. 3 shows a system for producing sugar according to the present invention;

FIGS. 4 to 7 shows various embodiments of gravity filter to be used in the said system according to the present invention.

FIG. 8 shows a settler/floater system according to one embodiment of the present invention to be used in the system of the present invention;

FIG. 9 a twin belt squeezer according to one embodiment of the present invention to be used in the system of the present invention;

FIGS. 10 to 14 show various embodiments of the pressure filters according to the present invention to be used in the system of the present invention.

### DETAILED DESCRIPTION OF THE INVENTION

The present invention is described more fully hereinafter with reference to the accompanying drawings, in which exemplary embodiments of the present invention are shown. The present invention may, however, be embodied in many different forms and should not be construed as limited to the exemplary embodiments set forth herein. Rather, these embodiments are provided so that this disclosure will be thorough and complete, and will fully convey the scope of the present invention to those skilled in the art. In the drawings, the sizes and relative sizes of layers and regions may be exaggerated for clarity.

Spatially relative terms, such as “above” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. The terminology used herein is for the purpose of describing particular embodiments only and is not intended to be limiting of the present invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Exemplary embodiments of the present invention are described herein with reference to cross-section illustrations that are schematic illustrations of idealized embodiments (and intermediate structures) of the present invention. As such, variations from the shapes of the illustrations as a result, for example, of manufacturing techniques and/or tolerances, are to be expected. Thus, exemplary embodiments of the present invention should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In general, the present invention provides a method and system for manufacturing clarified juice from sugarcane without using sulfitation process and with less heating. The method comprises step of steps of: washing sugarcane with water comprising biocide before passing through cutter and shredders for removing mud and reducing microbial population; spraying biocide on the cut and prepared sugarcane to reduce the microbial activity; adding biocide while milling of the sugarcane or in juice collectors to have control over the microbial growth; filtering the juice to remove floating/suspended matters; heating the filtered juice at about 35-85 deg C.; mixing one or more coagulating and flocculating agents for coagulation of suspended solids; and separating the coagulated solids to obtain clarified juice. This juice can be further treated to manufacture ethanol, juice for packaging having shelf life of about six months and sugar without using conventional sulfitation treatment.

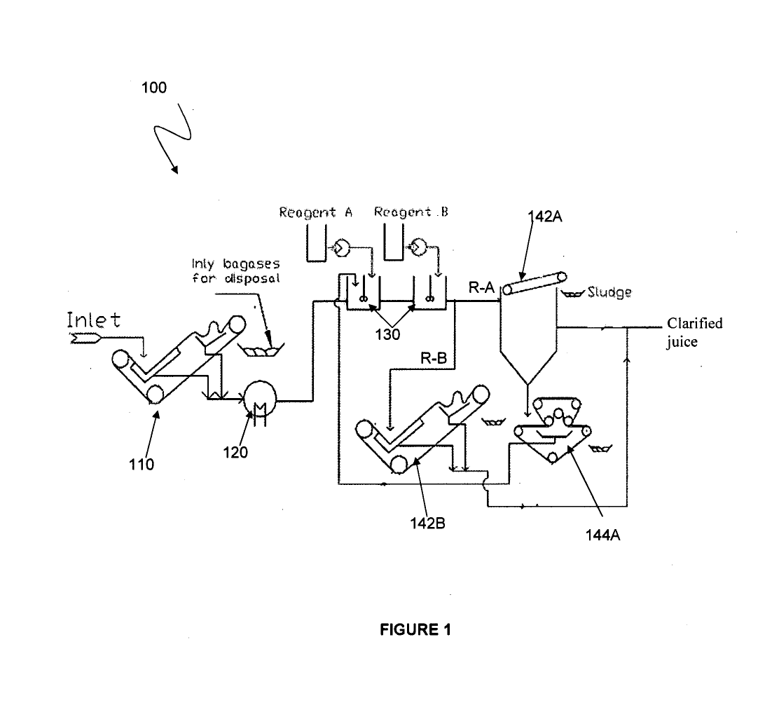

Referring FIG. 1 which shows a system (100) for producing a clarified juice from sugarcane according to the present invention comprising: a washing means (not shown) for washing sugarcane with water and biocide for reducing microbial population; cutter and shredders (not shown) for cutting and shredding the sugarcane; a spraying means (not shown) for spraying biocides on cut and shredded sugarcane for reducing microbial activity; series of mills (not shown) for extraction of juice form sugarcane; a means for adding biocide (not shown) in the juice during milling or in juice collectors; a gravity filter (110) for filtering the juice; a heater (120) for heating the filtered juice at about 35-85 deg C.; a mixer (130) for mixing coagulating agent and flocculating agent in the heated juice; and a separating means (140) for separating coagulated matters to obtain clarified juice. The FIG. 1 shows two alternative routes viz. Route A and Route B for passing the heated juice through the separating means (140) to obtain clarified juice. As shown in FIG. 1, according to Route A the separating means (140) involves a settler/floater system (142A) to obtain clarified juice and a filtering means (144A) for filtering settled mass of juice in order to re-circulate the filtrate. In Route B, the separating means (140) includes a gravity filter (142B) for obtaining clarified juice. Accordingly, a method for producing a clarified juice from sugarcane first comprises a step of washing sugarcane carried from farms with water comprising biocide before passing through cutter and shredders for removing mud and reducing microbial population. The biocide used in the water for washing the sugarcane is aqueous formulation of Formula I comprising single or a mixture of ammonium compound, gluteraldehyde or chlorine releasing compounds, hydrogen peroxide, peroxy acetic acid and water. The washing results in not only decrease in the microbial population but also moves the mud therefore decrease the load on the filters. The washed muddy water can be used for watering the farms.

In second step the washed sugarcane is then cut and shred by passing through the cutter and shredder in a known way and then biocide is sprayed on the cut and prepared sugarcane while moving over the belt to reduce the microbial activity. the biocide sprayed during cutting and shedding is aqueous formulation of Formula II comprising mixture of synergistically acting solutions of sodium and/or potassium salts of methyl, &/or dimethyl, &/or ethyl &/or cyanodiethyl dithiocarbamates about 25-50% w/w preferably 40±0.5% w/w; one or in combination of amine based, polymer based, phosphate based, phosphonate based, organosulfur based, quinine based chelating/sequestrating/penetrating compound about 0.01 to 5% w/w; and 0 to 10 parts of foaming or non-foaming type dispersant/chelating agent formulated to kill about 90% microbes in one minute.

In third step, the cut and shred sugarcane is then pass through the series of mills in a know way to extract juice from the sugarcane. According to the present invention biocide can be added while milling of the sugarcane or in juice collectors to have control over the microbial growth. The biocide added during milling or in the juice collector is aqueous formulation of Formula IIi comprising: a mixture of synergistically acting solutions of sodium and/or potassium salts of methyl, &/or dimethyl, &/or ethyl &/or cyanodiethyl dithiocarbamates about 25-50% w/w preferably 40±0.5% w/w, and 0 to 10 parts of foaming or non-foaming type dispersant/chelating agent formulated to kill about 90% microbes in ten minute. Then the juice is filtered to remove floating/suspended matters and; heated to about 35-85 deg C. for mixing one or more coagulating and flocculating agents for coagulation of suspended solids. This heated juice after mixing with coagulating and flocculating agents is separated from the coagulated solids to obtain clarified juice. The clarified juice obtained has negligible microbes less than 500 cfu per ml. According to the present invention, the step of separating coagulated solids involves filtering of juice or passing of juice through a floater/settler system to obtain clarified juice and filtering of settled mass of juice in settling tank of the floater/settler system to re-circulate the filtrate.

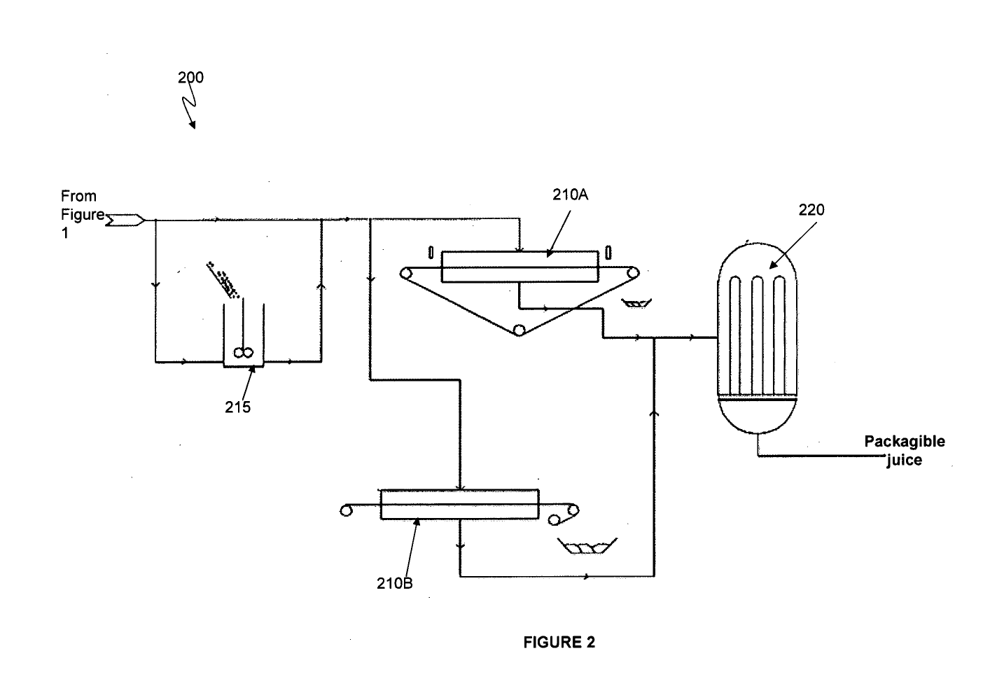

According to the present invention, a system for producing ethanol from sugarcane comprises a system for producing clarified juice of FIG. 1 and a fermenter for fermenting the clarified juice. Accordingly, the method for producing ethanol comprises a step of fermenting the juice produced by above method with the help of ethanol or yeast like Saccharomyces spp. or conventionally known microorganisms. Referring FIG. 2 shows a system (200) for producing ethanol. This system (200) includes all embodiments and routes of the system shown in FIG. 1 along with other embodiments to produce juice for packaging. This system (200) includes a system (100) for producing a clarified juice of FIG. 1, a filtering means and polishing filter (220). In alternative, the system may include a mixer (215) for addition of decolorizing and/or deodorizing agent before passing through filtering means (210). The filtering means (210) can be a gravity filter (210A) or a pressure filter (210B) for filtering the juice. The juice obtained from the polishing filter (220) comprises substantially zero microbes can be send for packaging for drinking purpose.

As shown in FIG. 2 of system for manufacturing sugarcane juice for packaging, the method for manufacturing a juice for packaging comprises steps of: filtering of the clarified juice obtained and pasteurizing and packaging the juice. According to the present invention, filtering of juice includes pressure filtration or long filtration of the juice. The juice thus obtained comprises substantially zero microbes can be send for packaging for drinking purpose and has shelf life about six months from the date of manufacturing.

According to the present invention, the method of manufacturing a juice for packaging may comprise addition of decolorizing and/or deodorizing agent to the juice for removal of color matter before filtering the juice. The decolorizing and/or deodorizing agent can be activated carbon, powdered or granular charcoal.

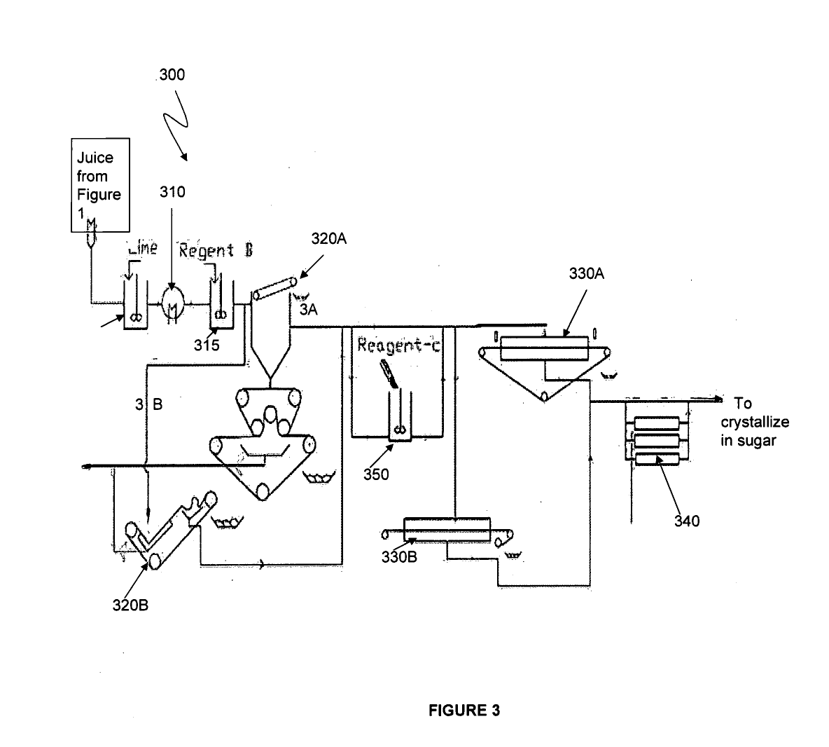

Referring FIG. 3 shows a system (300) for manufacturing sugar from sugarcane includes all embodiments and routes of the system shown in FIG. 1 along with other embodiments to treat the juice to manufacture sugar. Accordingly, the system (300) of manufacturing sugar from sugarcane includes a system (100) for producing a clarified juice of FIG. 1; a mixer (305) for raising the ph of the clarified juice obtained from the system of FIG. 1, a series of heater (310) for the heating juice at about 80-100 deg C.; a mixer (315) for mixing flocculating agents to flocculate proteins; a separating means (320) for separating the heated juice from flocculated proteins; a filtering means (330) for filtering the protein free juice; a concentrating means (340) for concentrating the juice; and a crystallizing means (not shown) for producing sugar.

In FIG. 3 the separating means (320) of the system for producing sugar shown is a settler/floater system (320A) by route 3A or a gravity filter (320B) by route 3B.

In optional route (OR-1), the system for producing sugar comprises a mixer (350) for addition of decolorizing and/or deodorizing agent before the filtering means (330).

According to the present invention, the filtering means (330) of the system for producing sugar is pressure filter (330B).

According to one embodiment of the system for producing sugar from sugarcane, the concentrating means (340) and crystallizing means can be a series of mesquite boiling scheme. According to another embodiment of the system for producing sugar from sugarcane, the concentrating means (340) is a reverse osmosis system or multiple effect evaporator system or evaporation system and the crystallizing means (not shown) is a series of pan boiling scheme to produce sugar.

Accordingly, a method for manufacturing sugar from the sugarcane comprises step of: adding lime in the clarified juice or the packagible juice to the raise the pH of the juice in the range of 6-9 heating the juice to flocculate proteins. Then the juice is passed through the separating means and filter means to separate floating matters and flocculated proteins. The clean juice obtained from the filtering means is then concentrated and crystallized to obtain sugar.

In an alternative, the method for manufacturing sugar from the sugarcane, the method may comprises a step of adding decolorizing and/or deodorizing agent in the juice obtained from the separating means; filtering the juice to obtain clear juice; passing the clear juice through the reverse-osmosis system to obtain concentrated juice; and thereafter crystallizing the juice into mesquite to obtain sugar.

According to the one embodiment of the invention, the step of separating juice from flocculated proteins involves passing of juice through gravity filter or twin belt filter. Alternatively, the separating involves passing of juice through a floater/settler system to obtain clarified juice and filtering of settled mass of juice in the settling tank to re-circulate the filtrate.

According to the one embodiment of the manufacturing sugar, filtering of the juice to obtain clear juice includes pressure filtration of the juice.

According to one embodiment of method for manufacturing sugar from the sugarcane, flocculating agents can be added for agglomeration of proteins flocculated.

The present invention also provides a settler/floater system, a gravity filter, and a pressure filter to be used for above systems and methods.

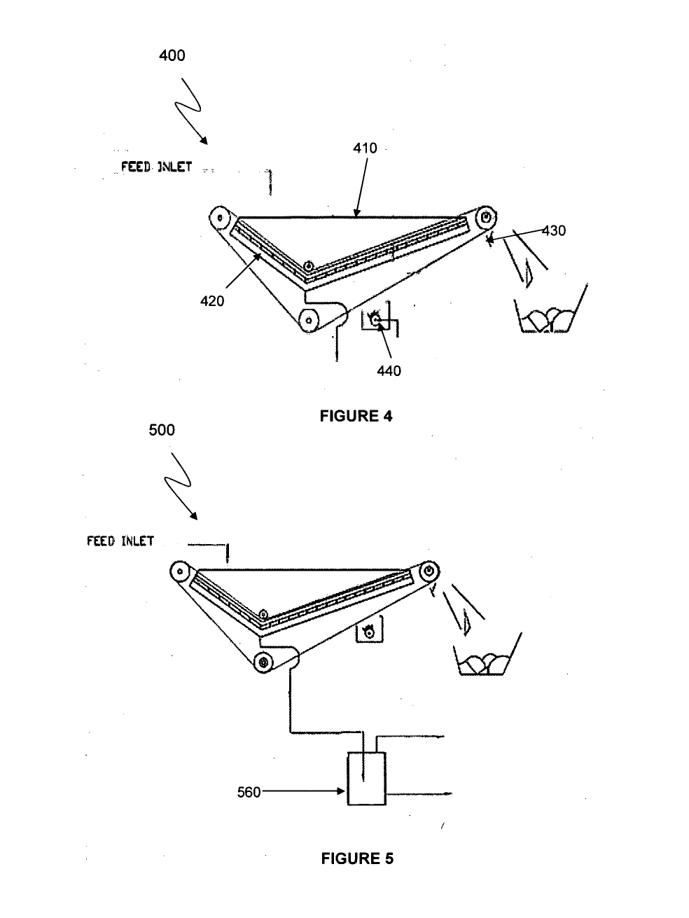

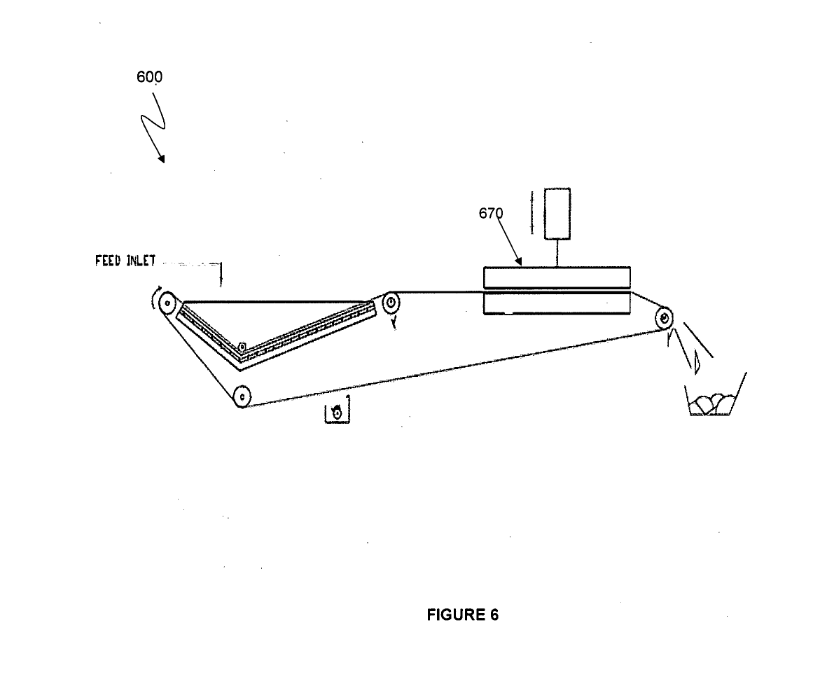

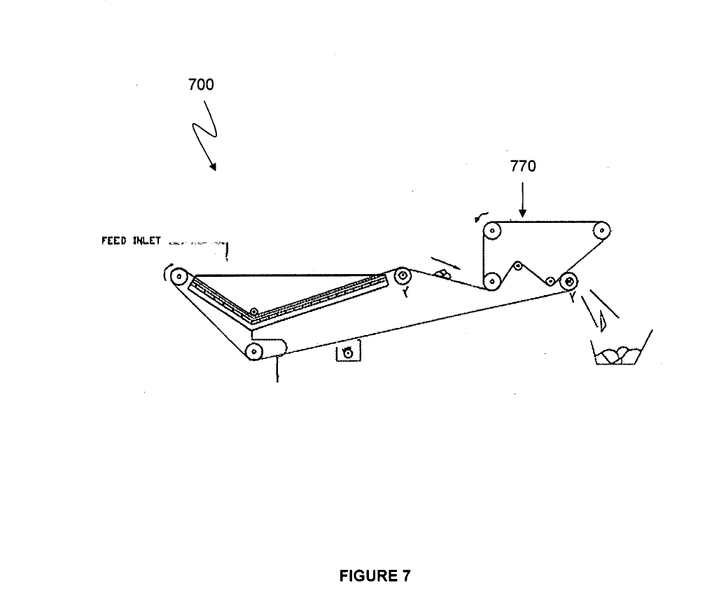

The gravity filters (400, 500, 600, 700) according to the present invention are shown through FIG. 4 to 7. The gravity filter (400, 500, 600, 700) comprises an endless filter cloth (410) rotating continuously or intermittently between atleast two rollers for filtration of juice, a feed bowl (420) to cause the belt to take shape of bowl, a scrapper (430) to remove solids deposited on the belt, a washing mean (440) to wash the belt before entering into bowl and a filtrate collecting tray (450) for collecting filtered juice. Referring to FIG. 5 wherein the gravity filter (500) comprises a vacuum means (560) arranged below the belt to increase speed of filtering. In FIGS. 6 and 7, the gravity filter (600, 700) comprise a squeezing means such as a plate squeezing means (670) and belt squeezing means (770) before the scrapper to squeeze solids deposited on the filter cloth.

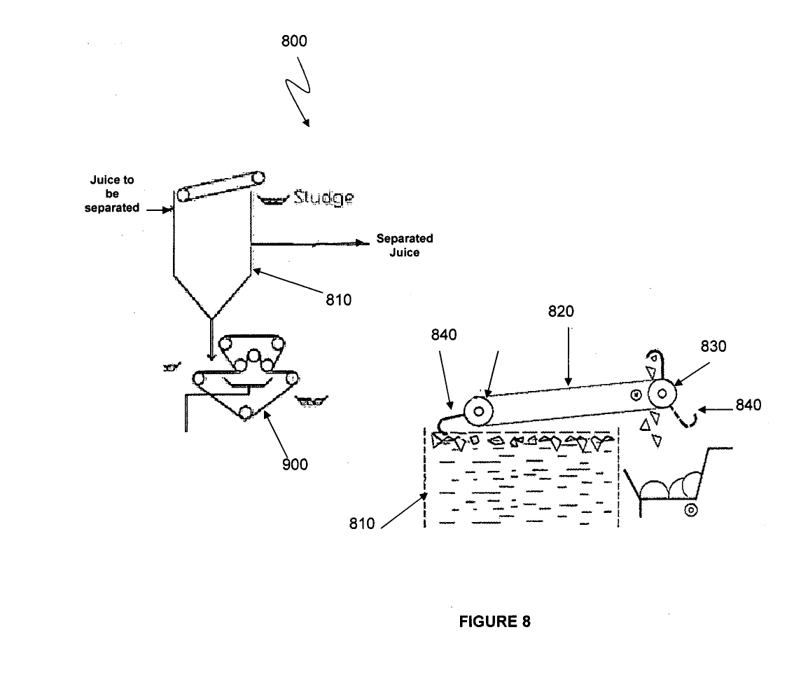

Referring to FIG. 8 shows the settler/floater system (800) according to the present invention. The settler/floater system (800) comprises a settling tank (810) for settling of the juice; an inclined endless belt (820) rotating between atleast two rollers (830) arranged at the top of the settling tank having a plurality of curve blades (840) adapted on the belt to collect the floating materials from the top of the liquid in the settling tank and to drop them at a predetermined point. A twin belt squeezer (900) is arranged below the settling tank for squeezing the slurry leaving the bottom of the settling tank to separate the solids from filtrate and re-circulating the filtrate and an outlet for removing clarified juice to obtain maximum recovery. According to the present invention, the settling tank can be a Dorr Settling tank. The curved blades (830) can be permeable or perforated blades for collecting floating matter from the top of the juice and allowing passage of juice through the blades.

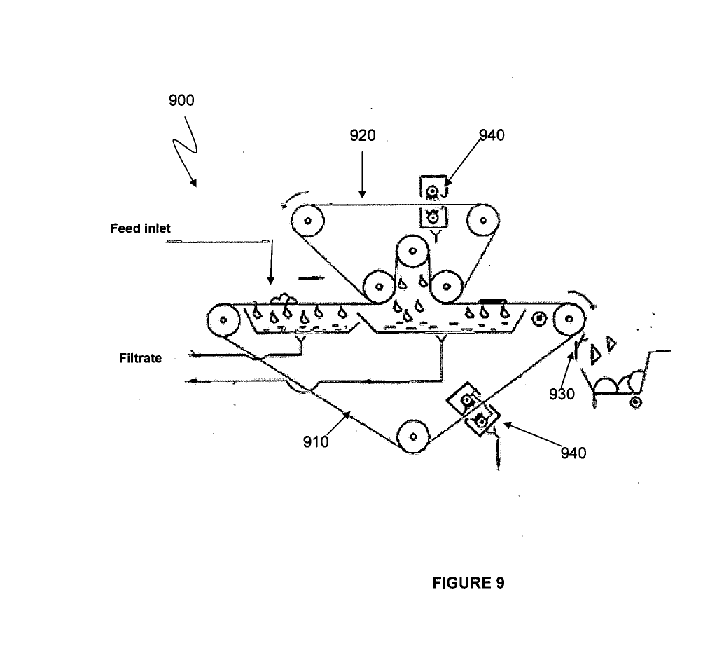

Referring to FIG. 9 shows the twin belt squeezer (900) comprises atleast two endless belts (910, 920) made of filter cloth and arranged one above another rotating in opposite directions having some portion in contact with each other to cause the slurry filtration between the cloths initially under gravity and by squeezing and shearing of the solids between the belts, a scrapper (930) for at least one belt to remove the solids deposited on the belt and a washing means (940) for each belt for washing the scrapped belt. The endless belt (910, 920) rotates at least between the two rollers and preferably more than two rollers to rotate the belts generally in serpentine manner.

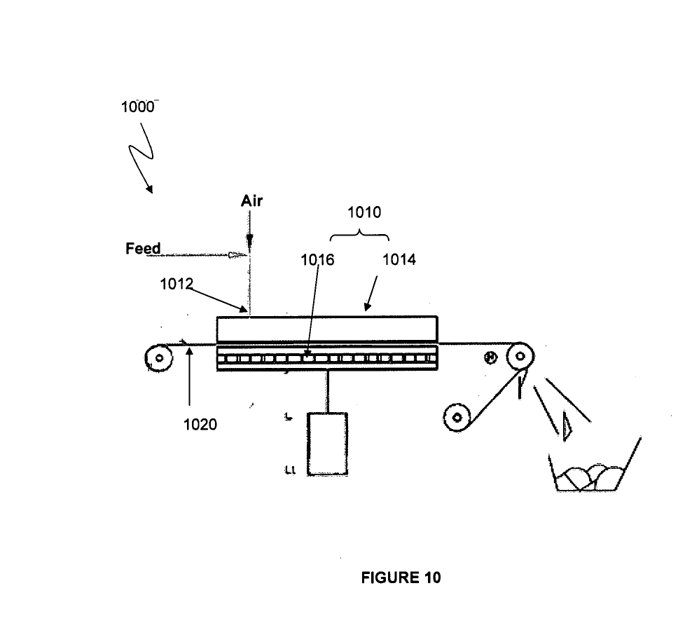

FIGS. 10 to 14 show various embodiments of the pressure filter (1000, 1100, 1200, 1400) according to the present invention.

According to the present invention, in general the pressure belt filter (1000, 1100, 1200, 1400) comprises: a closed filtration chamber (1010) having an inlet (1012) for feeding the juice to be filtered, a filter cloth (1020) dividing the filtration chamber (1010) into a feed chamber (1014) and a filtrate chamber (1016); a perforated support for supporting the filter cloth (not shown), and a pump (not shown) for supplying the juice at a high pressure. A vacuum means (not shown) can be connected to the filtrate chamber to increase the speed of the filtration.

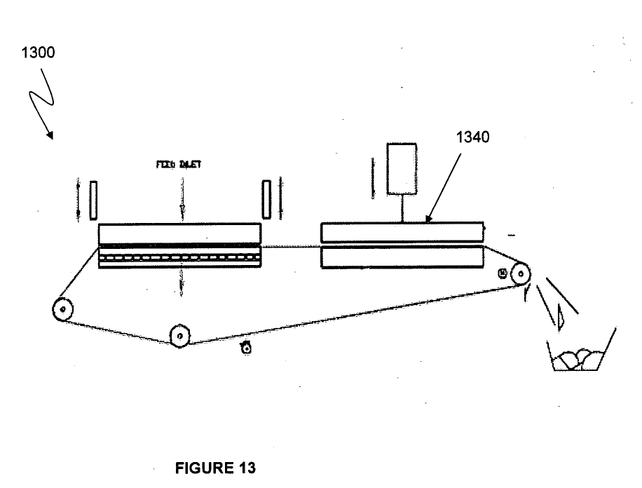

As shown in FIG. 12, the feed chamber (1214) and the filtrate chamber (1216) can be separate chambers and comprise a locking means (not shown)) for locking and unlocking the chambers to form a closed chamber.

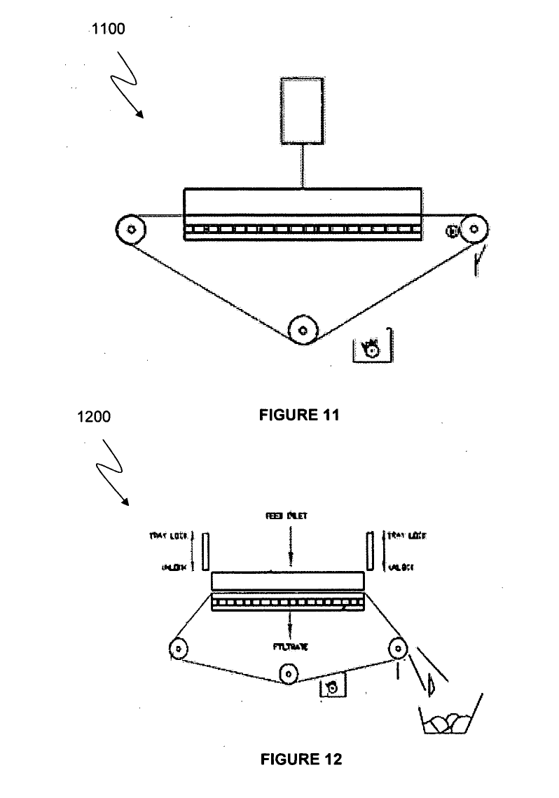

FIGS. 11-14 shows the filter cloth (1120, 1220, 1320) of belt system of the pressure filter (1100, 1200, 1400) can be arranged in an endless belt form rotating between atleast three rollers.

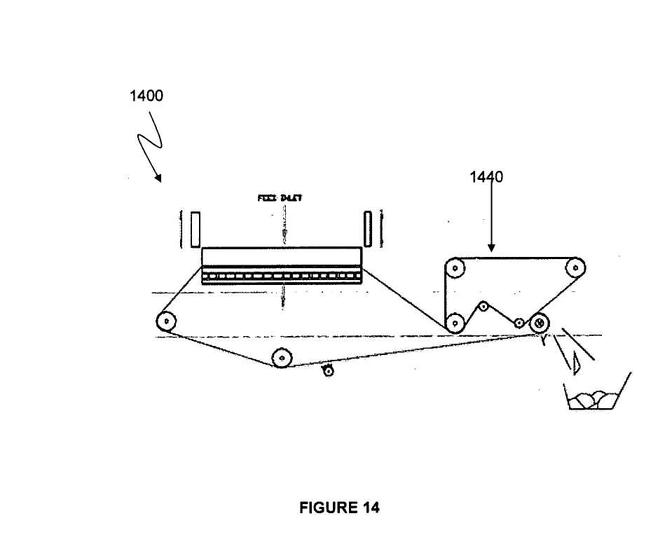

An alternative embodiment shown in FIGS. 13 and 14 comprises a pressure filter having a squeezing means for squeezing solids deposited on the filter cloth. The squeezing means can be a pressure plate squeezing means (1340) as shown in FIG. 13 or a belt squeezing means (1440) as shown in FIG. 14 for squeezing the solids or both.

The filter cloth according to the present invention may be woven/non-woven polyester, HDPE, polyamide or alike having pour size of 50 to 750 micron.

The present invention has made possible to pack the sugar-juice. The sugar obtained by this invention is refined sugar. The sugar produced have color less than 45 IU, conductivity ash below 0.15% and will have very low impurities like dextran, calcium, microorganism suspended particles etc and thus will satisfy the quality requirements of EU I and/or EU II grade sugar. Further, sugar obtained by the present invention contains substantially zero sulfur as the said method and system does not contain conventionally sulfitation treatment.

The foregoing is illustrative of the present invention and is not to be construed as limiting thereof. Although a few exemplary embodiments of the present invention have been described, those skilled in the art will readily appreciate that many modifications are possible without materially departing from the novel teachings and advantages of the present invention. Accordingly, all such modifications are intended to be included within the scope of the present invention as defined in the claims. In the claims, means-plus-function clauses are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein:

1. A method for producing clarified juice from sugarcane comprising steps of:
washing sugarcane with water having biocide wherein the biocide is aqueous formulation of Formula I comprising alone or a mixture of compound selected from a group of ammonium compound, gluteraldehyde or chlorine releasing compounds, hydrogen peroxide, and peroxy acetic acid;
passing washed sugarcane through cutter and shredders for removing mud and reducing microbial population;
spraying biocide on the cut and prepared sugarcane to reduce the microbial activity wherein the biocide is aqueous formulation of Formula II comprising mixture of synergistically acting solutions of sodium and/or potassium salts of methyl, &/or dimethyl, &/or ethyl &/or cyanodiethyl dithiocarbamates about 25-50% w/w preferably 40+0.5% w/w; one or in combination of amine based, polymer based, phosphate based, phosphonate based, organosulfur based, quinine based chelating/sequestrating/penetrating compound about 0.01 to 5% w/w; and 0 to 10 parts of foaming or non-foaming type dispersant/chelating agent formulated to kill about 90% microbes in one minute;
cutting and shedding the sugarcane;
adding biocide while milling of the sugarcane or in juice collectors to have control over the microbial growth wherein the biocide added during milling or in the juice collector is aqueous formulation of Formula III comprising: a mixture of synergistically acting solutions of sodium and/or potassium salts of methyl, &/or dimethyl, &/or ethyl &/or cyanodiethyl dithiocarbamates about 25-50% w/w preferably 40+0.5% w/w, and 0 to 10 parts of foaming or non-foaming type dispersant/chelating agent formulated to kill about 90% microbes in ten minute;
filtering the juice to remove floating/suspended matters;
heating the filtered juice at about 35-85 deg C.;
mixing one or more coagulating and flocculating agents for coagulation of suspended solids; and separating the coagulated solids to obtain clarified juice having negligible microbes less than 500 cfu per ml.

2. The method for producing clarified juice from sugarcane as claimed in claim 1, wherein the step of separating coagulated solids involves filtering of juice or passing the juice through a floater/settler system to obtain clarified juice and filtering of settled mass of juice in settling tank of the floater/settler system to re-circulate the filtrate.

3. The method for producing clarified juice from sugarcane as claimed in claim 1, wherein the coagulating agent is selected from a group of alumina compounds, alum, poly-aluminum chloride or in combination thereof.

4. The method for producing clarified juice from sugarcane as claimed in claim 1, wherein the flocculating agent is selected from a group of poly-electrolyte, polymers or co-polymers of acryl amide acrylic acid malic acid or the like and in combination thereof.

5. A method for producing ethanol comprising steps of fermenting the clarified juice obtained from the method as claimed in claim 1 by adding fermenting agents such as ethanol, yeast e.g. Saccharomyces spp etc.

6. A method for manufacturing juice for packaging comprises steps of: filtering of clarified juice obtained from the method for producing clarified juice from sugarcane as claimed in claim 1; and
pasteurizing and packaging the juice wherein the juice has shelf life about six month as the microbial activity is substantially equal to zero.

7. The method for manufacturing juice for packaging as claimed in claim 6, filtering of juice includes pressure filtration or long filtration of the juice.

8. The method of manufacturing a juice for packaging as claimed in claim 6 wherein decolorizing and/or deodorizing agent can be added to the juice for removal of color matter before filtering the juice.

9. The method of manufacturing a juice for packaging as claimed in claim 8, wherein the decolorizing and/or deodorizing agent is activated carbon, powdered or granular charcoal.

10. A method for manufacturing sugar from the sugarcane comprises steps of:
adding lime in the clarified juice obtained from the method as claimed in claim 1 to raise the pH of the juice in the range of 6-9;
heating the juice to flocculate proteins;
mixing know reagents to flocculate proteins;
separating the juice from flocculated proteins;
concentrating protein-free juice through settler floater to remove suspended and floating matters; and
crystallizing the juice to obtain sugar.

11. The method for manufacturing sugar from the sugarcane as claimed in claim 10 further comprises steps of:
adding decolorizing and/or deodorizing agent in the juice separated;
filtering the juice to obtain clear juice;
passing the clear juice through the reverse-osmosis system or thru multiple effect evaporation system to obtain concentrated juice before crystallizing the juice into mesquite to obtain sugar.

12. The method for manufacturing sugar from the sugarcane as claimed in claim 10, wherein the step of separating juice from flocculated proteins involves passing of juice through gravity filter or twin belt filter or through a floater/settler system to obtain clarified juice and filtering of settled mass of juice in the settling tank to re-circulate the filtrate.

13. The method for manufacturing sugar from the sugarcane as claimed in claim 10, wherein filtering of the juice to obtain clear juice includes pressure filtration of the juice.

14. The method for manufacturing sugar from the sugarcane as claimed in claim 10, wherein flocculating agents can be added for agglomeration of proteins flocculated.

15. A system for producing a clarified juice from sugarcane comprising:
a washing means for washing sugarcane with water and biocide for reducing microbial population;
a spraying means for spraying biocides solution on prepared cane before entry to cutter and/or shredder for reducing microbial activity; cutter and shredders for cutting and shredding the sugarcane;
a series of mills for extraction of juice form sugarcane;
a means for adding biocide in the juice during milling or in juice collectors of the mills;
a gravity filter for filtering the juice; a heater for heating the filtered juice at about 35-85 deg C.;
a mixer for mixing coagulating agent and flocculating agent in the heated juice; and
a separating means for separating coagulated matters to obtain clarified juice wherein said clarified juice negligible microbes less than 500 cfu per ml.

16. The system for producing a clarified juice from sugarcane as claimed in claim 15, wherein a separating means includes a gravity filter for obtaining clarified juice or a settler/floater system to obtain clarified juice' and a filtering means for filtering settled mass of juice to re-circulate the filtrate.

17. A system for producing juice for packaging comprising a system for producing a clarified juice as claimed in claim 15; a filter for filtering the clarified juice; and pasteurizing and packaging means for the filtered juice.

18. The system for producing juice for packaging as claimed in claim 17, wherein the filter may be a polishing filter.

19. The system for producing juice for packaging as claimed in claim 17 further comprises
a mixer for addition of decolorizing and/or deodorizing agent; and
a pressure filter for filtering the juice before passing the juice through polishing filter.

20. A system for producing sugar from sugarcane comprising:
a system for producing a clarified juice as claimed in claim 15;
a series of heater for the heating juice at about 80-100 deg C.;
a separating means for separating the heated juice from flocculated proteins;
a filtering means for filtering the protein free juice;
a concentrating means for concentrating the juice; and
a crystallizing means for producing sugar.

21. The system for producing sugar from sugarcane as claimed in claim 20, wherein the separating means of the system is a settler/floater system or a gravity filter.

22. The system for producing sugar from sugarcane as claimed in claim 20 further comprises a mixer for addition of decolorizing and/or deodorizing agent before the filtering means.

23. The system for producing sugar from sugarcane as claimed in claim 20, wherein the filtering means of the system for producing sugar is pressure filter.

24. The system for producing sugar from sugarcane as claimed in claim 20, wherein, the concentrating means is a reverse osmosis system or multiple effect evaporator system or evaporation system and the crystallizing means is a series of pan boiling scheme to produce sugar.

25. A system for producing ethanol from sugarcane comprising:
a system for producing a clarified juice as claimed in claim 15; and
a fermenting device.

26. The system for producing a clarified juice from sugarcane as claimed in claim 15, wherein the settler/floater system comprises a settling tank for settling of the juice; an inclined endless belt rotating between two rollers arranged at the top of the settling tank having a plurality of curve blades adapted on the belt to collect the floating materials from the top of the liquid in the settling tank and to drop them at a predetermined point; a twin belt squeezer for squeezing the slurry leaving the bottom of the settling tank to separate the solids from filtrate and re-circulating the filtrate and an outlet for removing clarified juice.

27. The system for producing a clarified juice from sugarcane as claimed in claim 26, wherein the curved blades are permeable or perforated blades for collecting floating matter from the top of the juice and allowing passage of juice through the blades.

28. The system for producing a clarified juice from sugarcane as claimed in claim 26, the twin belt squeezer comprises two endless belts made of filter cloth and arranged one above another rotating in opposite directions to cause the slurry filtration between the cloths initially under gravity and further by squeezing and shearing of the solids between the belts, a scrapper for a belt to remove the solids deposited on the belt and a washing means for each belt for washing the scrapped belt.

29. The system for producing a clarified juice from sugarcane as claimed in claim 15, wherein the gravity filter comprises an endless filter cloth rotating continuously or intermittently between two rollers for filtration of juice, a feed bowl to cause the belt to take shape of bowl, a scrapper to remove solids deposited on the belt, a washing mean to wash the belt before entering into bowl and a filtrate collecting tray for collecting filtered juice.

30. The system for producing a clarified juice from sugarcane as claimed in claim 29, wherein the gravity filter may comprise a squeezing means such as a belt squeezing/plate squeezing means before the scrapper to squeeze solids deposited on the filter cloth.

31. The system for producing a packaging juice from sugarcane as claimed in claim 17, wherein the pressure belt filter comprises: a closed filtration chamber having an inlet for feeding the juice to be filtered, a filter cloth dividing the filtration chamber into feed chamber and filtrate chamber; a perforated support for supporting the filter cloth, a squeezing means for squeezing solids deposited on the filter cloth and a pump for supplying the juice at a high pressure.

32. The system for producing a packaging juice from sugarcane as claimed in claim 31, wherein the closed chamber may comprise a feed chamber and a filtrate chamber separable from each other and a locking means for locking and unlocking the chambers.

33. The system for producing a packaging juice from sugarcane as claimed in claim 31, the filter cloth may arranged in an endless belt form rotating between two rollers and preferably between more than two rollers intermittently.

34. The system for producing a packaging juice from sugarcane as claimed in claim 31, the squeezing means may comprises a means to supply air at high pressure or a belt squeezing means for squeezing the solids or both.

35-38. (canceled)
